(19) United States
(12) Patent Application Publication
(10) Pub. No.: US 2017/0304123 A1
(43) Pub. Date: Oct. 26, 2017

(54) ABSORBENT ARTICLES COMPRISING PATTERNED NONWOVEN MATERIALS

(71) Applicant: The Procter & Gamble Company, Cincinnati, OH (US)

(72) Inventors: John FERRER, Mason, OH (US); Han XU, Cincinnati, OH (US)

(21) Appl. No.: 15/646,120

(22) Filed: Jul. 11, 2017

Related U.S. Application Data
(63) Continuation of application No. 13/956,435, filed on Aug. 1, 2013.
(60) Provisional application No. 61/678,159, filed on Aug. 1, 2012.

(51) Int. Cl. A61F 13/15; A61F 13/514; A61F 13/515; A61F 13/84; A61F 13/513

(52) U.S. Cl. CPC A61F 13/15203; A61F 13/84; A61F 13/51496; A61F 13/51394; A61F 13/515; A61F 2013/15243; A61F 2013/8497

(57) ABSTRACT

An absorbent article comprising a topsheet comprising a first nonwoven material having a first visible element, a backsheet comprising a second nonwoven material having a second visible element, and an absorbent core disposed between the topsheet and the backsheet, wherein the first visible element and the second visible element are matching visible elements. Matching visible elements may enhance a caregiver's understanding of breathability and air flow between absorbent article components by indicating a pathway for air to flow.

### FIELD OF THE INVENTION

The present invention relates to absorbent articles comprising patterned nonwoven materials on the interior and/or exterior and/or additional components of the product.

### BACKGROUND OF THE INVENTION

Infants and other individuals wear disposable absorbent articles such as diapers or feminine care pads to receive and contain urine and other body exudates. Much work has been done to create visual patterns on the inner liner of the absorbent articles to communicate holistic design themes to the consumer, such as examples of increasing softness and breathability levels for skin health benefit. Actual breathability is known as a function of the full absorbent article design, and all elements of the absorbent article must work together to enhance the breathability performance of the product. Currently, absorbent articles comprise materials of various levels of air and water vapor permeability to aid in the heat/air exchange of the diapers. However, it is difficult to convey to consumers the breathability benefit they receive, as the function is not visible to the eye. Additionally, as there are different types of materials in the absorbent article, there is not clarity as to how the materials work together.

Thus, there is a need for a disposable absorbent article that visually coordinates the materials in the products so that design features such as breathability and softness of the materials is more apparent to consumers. There is also a need to coordinate patterns on different elements of the articles to create an overall more pleasing article to the consumer.

### SUMMARY OF THE INVENTION

In one embodiment, the present invention is directed to an absorbent article comprising a topsheet comprising a first nonwoven material having a first visible element, a backsheet comprising a second nonwoven material having a second visible element, and an absorbent core disposed between the topsheet and the backsheet, wherein the first visible element and the second visible element are matching visible elements.

In one embodiment, the present invention is directed to an absorbent article comprising a topsheet comprising a first nonwoven material, a backsheet comprising a second nonwoven material, an absorbent core disposed between the topsheet and the backsheet, and a leg cuff material comprising a third nonwoven material, wherein at least two of the first nonwoven material, the second nonwoven material, and the third nonwoven material have matching visible elements. In another embodiment of the present invention, the first nonwoven material, the second nonwoven material, and the third nonwoven material all have matching visible elements.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a perspective view of an absorbent article shown laid out horizontally in a relaxed condition, wearer-facing surfaces up;

FIG. 2 is a plan view of an absorbent article shown laid out horizontally in a stretched out, flattened state (stretched out against elastic contraction induced by the presence of elastic members), wearer-facing surfaces facing the viewer;

FIG. 3 is a cross section of the the absorbent article depicted in FIGS. 1 and 2, taken through line 2-2 in those figures;

FIG. 4 is a schematic cross section view of an example of an absorbent core suitable in one embodiment of the invention;

FIG. 5 is a schematic cross section view of another example of an absorbent core suitable in one embodiment of the invention;

FIG. 6 is a schematic cross section view of another example of an absorbent core suitable in one embodiment of the invention;

FIG. 7 is a perspective view of an exemplary absorbent article shown laid out horizontally in a relaxed condition, wearer-facing surfaces up;

FIG. 8 is an exemplary visible element suitable in one embodiment of the invention;

FIG. 9 is an exemplary visible element suitable in one embodiment of the invention;

FIG. 10 is an exemplary visible element suitable in one embodiment of the invention.

### DETAILED DESCRIPTION OF THE INVENTION

As used herein, “absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments and pads, feminine hygiene pads, breast pads, care mats, bibs, wound dressing products, and the like. As used herein, the term “exudates” includes, but is not limited to, urine, blood, vaginal discharges, breast milk, sweat and fecal matter.

As used herein, “absorbent core” means a structure typically disposed between a topsheet and backsheet of an absorbent article for absorbing and containing liquid received by the absorbent article. The absorbent core may also include a cover layer or envelope. The cover layer or envelope may comprise a nonwoven. In some examples, the absorbent core may include one or more substrates, an absorbent polymer material, and a thermoplastic adhesive material/composition adhering and immobilizing the absorbent polymer material to a substrate, and optionally a cover layer or envelope.

“Absorbent polymer material,” “absorbent gelling material,” “AGM,” “superabsorbent,” and “superabsorbent material” are used herein interchangeably and refer to cross linked polymeric materials that can absorb at least 5 times their weight of an aqueous 0.9% saline solution as measured using the Centrifuge Retention Capacity test (Edana 441.2-01).

“Absorbent particulate polymer material” is used herein to refer to an absorbent polymer material which is in particulate form so as to be flowable in the dry state.

“Absorbent particulate polymer material area” as used herein refers to the area of the core wherein the first substrate and second substrate are separated by a multiplicity of superabsorbent particles. There may be some extraneous superabsorbent particles outside of this area between the first substrate 64 and second substrate.

“Airfelt” is used herein to refer to comminuted wood pulp, which is a form of cellulosic fiber.

As used herein, “disposable” is used in its ordinary sense to mean an article that is disposed or discarded after a limited number of usage events over varying lengths of time, for example, less than about 20 events, less than about 10 events, less than about 5 events, or less than about 2 events.

As used herein, “diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pant” which is defined below.

As used herein, “fiber” and “filament” are used interchangeably.

As used herein, “film”—means a skin-like or membrane-like layer of material formed of one or more polymers, which does not have a form consisting predominately of a web-like structure of consolidated polymer fibers and/or other fibers.

As used herein, a “nonwoven” is a manufactured sheet or web of directionally or randomly oriented fibers which are first formed into a batt and then consolidated and bonded together by friction, cohesion, adhesion or one or more patterns of bonds and bond impressions created through localized compression and/or application of pressure, heat, ultrasonic or heating energy, or a combination thereof. The term does not include fabrics which are woven, knitted, or stitch-bonded with yarns or filaments. The fibers may be of natural or man-made origin and may be staple or continuous filaments or be formed in situ. Commercially available fibers have diameters ranging from less than about 50 nanometers to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes including but not limited to meltblowing, spunbonding, spunmelting, solvent spinning, electrospinning, carding, film fibrillation, melt-film fibrillation, airlaying, dry-laying, wetlaying with staple fibers, and combinations of these processes as known in the art. The basis weight of nonwoven fabrics is usually expressed in grams per square meter (gsm).

Spunbonding includes the step of calender-bonding a batt of spunlaid fibers, to consolidate them and bond them together to some extent to create the web as a fabric-like structure and enhance mechanical properties e.g., tensile strength, which may be desirable so the material can sufficiently maintain structural integrity and dimensional stability in subsequent manufacturing processes, and in the final product in use. Calender-bonding may be accomplished by passing the batt through the nip between a pair of rotating calender rollers, thereby compressing and consolidating the fibers to form a nonwoven web. One or both of the rollers may be heated, so as to promote heating, plastic deformation, intermeshing and/or thermal bonding/fusion between superimposed fibers compressed at the nip. The rollers may form operable components of a bonding mechanism in which they are urged together by a controllable amount of force, so as to exert the desired compressing force/pressure at the nip. In some processes an ultrasonic energy source may be included in the bonding mechanism so as to transmit ultrasonic vibration to the fibers, again, to generate heat energy within them and enhance bonding.

One or both of the rollers may have their circumferential surfaces machined, etched, engraved or otherwise formed to have thereon a bonding pattern of bonding protrusions and recessed areas, so that bonding pressure exerted on the batt at the nip is concentrated at the bonding surfaces of the bonding protrusions, and is reduced or substantially eliminated at the recessed areas. The bonding surfaces have bonding surface shapes. As a result, an impressed pattern of bonds between fibers forming the web, having bond impressions and bond shapes corresponding to the pattern and bonding surface shapes of the bonding protrusions on the roller, is formed on the nonwoven web. One roller such as roller may have a smooth, unpatterned cylindrical surface so as to constitute an anvil roller, and the other roller may be formed with a pattern as described, to constitute a bonding pattern roller; this combination of rollers will impart a pattern on the web reflecting the pattern on the bonding pattern roller. In some examples both rollers may be formed with patterns, and in particular examples, differing patterns that work in combination to impress a combination pattern on the web such as described in, for example, U.S. Pat. No. 5,370,764.

As used herein, “pant” or “training pant”, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”. Suitable pants are disclosed in U.S. Pat. No. 5,246,433, issued to Hasse et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al. on Sep. 28, 1999.

“Substantially cellulose free” is used herein to describe an article, such as an absorbent core, that contains less than 10% by weight cellulosic fibers, less than 5% cellulosic fibers, less than 1% cellulosic fibers, no cellulosic fibers, or no more than an immaterial amount of cellulosic fibers. An immaterial amount of cellulosic material would not materially affect the thinness, flexibility, or absorbency of an absorbent core.

“Substantially continuously distributed” as used herein indicates that within the absorbent particulate polymer material area, the first substrate 64 and second substrate 72 are separated by a multiplicity of superabsorbent particles. It is recognized that there may be minor incidental contact areas between the first substrate 64 and second substrate 72 within the absorbent particulate polymer material area. Incidental contact areas between the first substrate 64 and second substrate 72 may be intentional or unintentional (e.g. manufacturing artifacts) but do not form geometries such as pillows, pockets, tubes, and the like.

“Visible element” as used herein is the unit cell on a component of an absorbent article having an Lmax dimension of greater than about 3.0 mm and a Wmax dimension of greater than about 0.3 mm, wherein Lmax is the greatest measureable length of the visible element measured from the outer edge of the visible element to the opposite outer edge of the visible element; and Wmax is the greatest measureable width perpendicular to Lmax and measured from an outer edge of the visible element to the opposite outer edge of the visible element; the visible element may be comprised of solid lines or dotted lines. “Unit cell” as used herein is the smallest building block of a pattern, whose geometric arrangement defines pattern's characteristic symmetry and whose repetition in space can re-construct the entire pattern. The visible element may be formed by printing, embossing, thermobonding, calendar-bonding, combinations thereof, or any other method of indicating a visual signal on an absorbent article component.

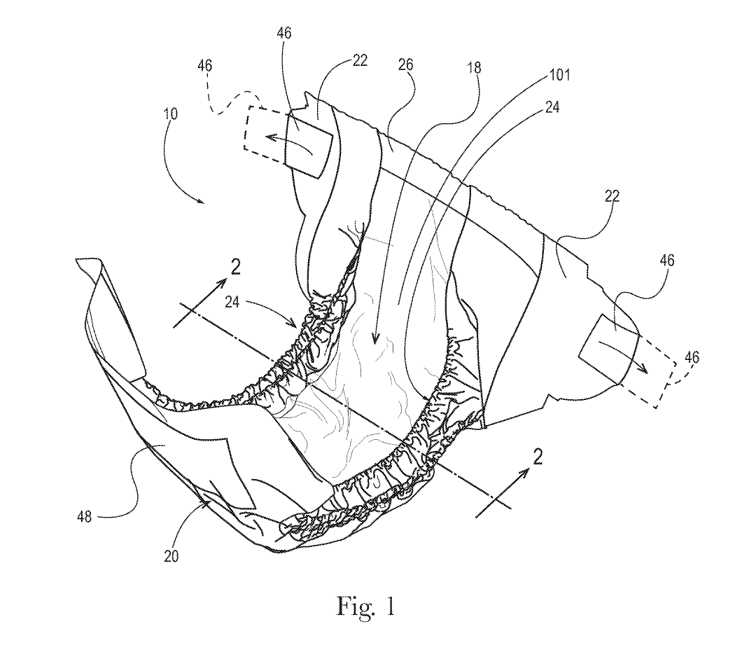

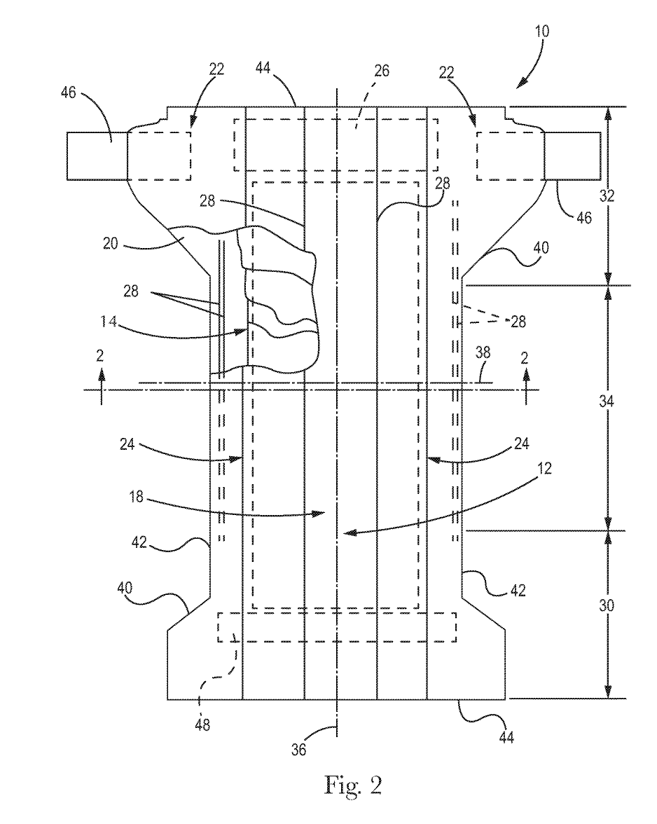

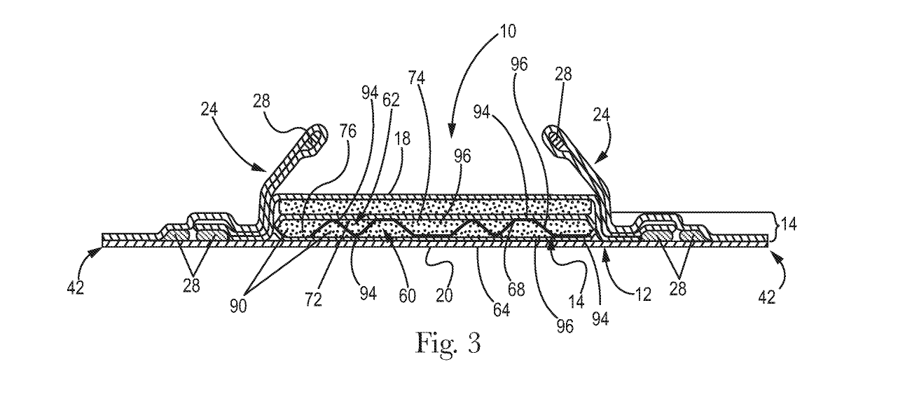

FIG. 1 is a perspective view of a diaper 10 in a relaxed, laid-open position as it might appear opened and lying on a horizontal surface. FIG. 2 is a plan view of a diaper 10 shown in a flat-out, uncontracted state (i.e., without elastic induced contraction), shown with portions of the diaper 10 cut away to show underlying structure. The diaper 10 is depicted in FIG. 2 with its longitudinal axis 36 and its lateral axis 38. Portions of the diaper 10 that contact a wearer are shown oriented upwards in FIG. 1, and are shown facing the viewer in FIG. 2. FIG. 3 is a cross section of the diaper taken at line 2-2 in FIGS. 1 and 2.

The diaper 10 generally may comprise a chassis 12 and an absorbent core 14 disposed in the chassis. The chassis 12 may comprise the main body of the diaper 10.

The chassis 12 may include a topsheet 18, generally on the wearer-facing surface of the absorbent article, which may be liquid pervious, and a backsheet 20, generally on the garment-facing surface of the absorbent article, which may be liquid impervious. The absorbent core 14 may be encased between the topsheet 18 and the backsheet 20. The chassis 12 may also include side panels 22, elasticized leg cuffs 24, and an elastic waist feature 26. The chassis 12 may also comprise a fastening system, which may include at least one fastening member 46 and at least one landing zone 48. One or more layers of the topsheet and/or backsheet may be formed of a nonwoven web as described below.

The leg cuffs 24 and the elastic waist feature 26 may each typically comprise elastic members 28. One end portion of the diaper 10 may be configured as a first waist region 30 of the diaper 10. An opposite end portion of the diaper 10 may be configured as a second waist region 32 of the diaper 10. An intermediate portion of the diaper 10 may be configured as a crotch region 34, which extends longitudinally between the first and second waist regions 30 and 32. The crotch region 34 may include from 33.3% to 50% of the overall length of the diaper 10, and each of waist regions 30, 32 may correspondingly include from 25% to 33.3% of the overall length of the diaper 10.

The waist regions 30 and 32 may include elastic elements such that they gather about the waist of the wearer to provide improved fit and containment (elastic waist feature 26). The crotch region 34 is that portion of the diaper 10 which, when the diaper 10 is worn, is generally positioned between the wearer's legs.

The diaper 10 may also include such other features including front and rear ear panels, waist cap features, elastics and the like to provide better fit, containment and aesthetic characteristics. Such additional features are described in, e.g., U.S. Pats. Nos. 3,860,003 and 5,151,092.

In order to apply and keep diaper 10 in place about a wearer, the second waist region 32 may be attached by the fastening member 46 to the first waist region 30 to form leg opening(s) and an article waist. When fastened, the fastening system carries a tensile load around the article waist.

According to some examples, the diaper 10 may be provided with a re-closable fastening system or may alternatively be provided in the form of a pant-type diaper. When the absorbent article is a diaper, it may comprise a re-closable fastening system joined to the chassis for securing the diaper to a wearer. When the absorbent article is a pant-type diaper, the article may comprise at least two side panels joined to the chassis and to each other to form a pant. The fastening system and any component thereof may include any material suitable for such a use, including but not limited to plastics, films, foams, nonwoven, woven, paper, laminates, stretch laminates, activated stretch laminates, fiber reinforced plastics and the like, or combinations thereof. In some examples, the materials making up the fastening device may be flexible. In some examples, the fastening device may comprise cotton or cotton-like materials for additional softness or consumer perception of softness. The flexibility may allow the fastening system to conform to the shape of the body and thus, reduce the likelihood that the fastening system will irritate or injure the wearer's skin.

For unitary absorbent articles, the chassis 12 and absorbent core 14 may form the main structure of the diaper 10 with other features added to form the composite diaper structure. While the topsheet 18, the backsheet 20, and the absorbent core 14 may be assembled in a variety of well-known configurations, preferred diaper configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999.

The topsheet 18 may be fully or partially elasticized and/or may be foreshortened to create a void space between the topsheet 18 and the absorbent core 14. Exemplary structures including elasticized or foreshortened topsheets are described in more detail in U.S. Pat. No. 5,037,416 entitled “Disposable Absorbent Article Having Elastically Extensible Topsheet” issued to Allen et al. on Aug. 6, 1991; and U.S. Pat. No. 5,269,775 entitled “Trisection Topsheets for Disposable Absorbent Articles and Disposable Absorbent Articles Having Such Trisection Topsheets” issued to Freeland et al. on Dec. 14, 1993.

The backsheet 20 may be joined with the topsheet 18. The backsheet 20 may serve to prevent the exudates absorbed by the absorbent core 14 and contained within the diaper 10 from soiling other external articles that may contact the diaper 10, such as bed sheets and clothing. Referring to FIG. 2B, the backsheet 20 may be substantially impervious to liquids (e.g., urine) and may be formed of a laminate of a nonwoven 21 and a thin polymeric film 23 such as a thermoplastic film having a thickness of about 0.012 mm (0.5 mil) to about 0.051 mm (2.0 mils). Nonwoven 21 may be a nonwoven web as described herein. Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the diaper 10 while still preventing liquid exudates from passing through the backsheet 20. Exemplary breathable materials may include materials such as woven webs, nonwoven webs, composite materials such as film-coated nonwoven webs, and microporous films such as manufactured by Mitsui Toatsu Co., of Japan under the designation ESPOIR and by EXXON Chemical Co., of Bay City, Tex., under the designation EXXAIRE. Suitable breathable composite materials comprising polymer blends are available from Clopay Corporation, Cincinnati, Ohio under the name HYTREL blend P1 8-3097. Other examples of such breathable composite materials are described in greater detail in PCT Application No. WO 95/16746, published on Jun. 22, 1995 in the name of E. I. DuPont. Other breathable backsheets including nonwoven webs and apertured formed films are described in U.S. Pat. No. 5,571,096 issued to Dobrin et al. on Nov. 5, 1996.

In some examples, the backsheet of the present invention may have a water vapor transmission rate (WVTR) of greater than about 2,000 g/24 h/m2, greater than about 3,000 g/24 h/m2, greater than about 5,000 g/24 h/m2, greater than about 6,000 g/24 h/m2, greater than about 7,000 g/24 h/m2, greater than about 8,000 g/24 h/m2, greater than about 9,000 g/24 h/m2, greater than about 10,000 g/24 h/m2, greater than about 11,000 g/24 h/m2, greater than about 12,000 g/24 h/m2, greater than about 15,000 g/24 h/m2, measured according to WSP 70.5 (08) at 37.8 0 C and 60% Relative Humidity.

Suitable nonwoven web materials useful in the present invention include, but are not limited to spunbond, meltblown, spunmelt, solvent-spun, electrospun, carded, film fibrillated, melt-film fibrillated, air-laid, dry-laid, wet-laid staple fibers, and other and other nonwoven web materials formed in part or in whole of polymer fibers, as known in the art. A suitable nonwoven web material may also be an SMS material, comprising a spunbonded, a melt-blown and a further spunbonded stratum or layer or any other combination of spunbonded and melt-blown layers, such as a SMMS or SSMMS etc. Examples include one or more layers of fibers with diameters below 1 micron (nanofibers and nanofiber layers); examples of these rise in combinations of SMS, SMNS, SSMNS or SMNMS nonwoven webs (where “N” designates a nanofiber layer). In some examples, permanently hydrophilic non-wovens, and in particular, nonwovens with durably hydrophilic coatings may be desirable. Typically, the suitable non-woven is air permeable. Typically the suitable nonwoven is water or liquid permeable, but may also be water impermeable by reason of fiber size and density, and hydrophobicity of the fibers. Water or liquid permeability may be enhanced by treatments to render the fibers hydrophilic, as discussed below.

The nonwoven web may be formed predominately of polymeric fibers. In some examples, suitable non-woven fiber materials may include, but are not limited to polymeric materials such as polyolefins, polyesters, polyamide, or specifically polypropylene (PP), polyethylene (PE), poly-lactic acid (PLA), polyethylene terephthalate (PET) and/or blends thereof. Nonwoven fibers may be formed of, or may include as additives or modifiers, components such as aliphatic polyesters, thermoplastic polysaccharides, or other biopolymers (bio-based or renewable polymers).

The individual fibers may be monocomponent or multicomponent. The multicomponent fibers may be bicomponent, such as in a core-and-sheath or side-by-side arrangement. Often, the individual components comprise aliphatic polyolefins such as polypropylene or polyethylene, or their copolymers, aliphatic polyesters, thermoplastic polysaccharides or other biopolymers.

Further useful nonwovens, fiber compositions, formations of fibers and nonwovens and related methods are described in U.S. Pat. No. 6,645,569 to Cramer et al., U.S. Pat. No. 6,863,933 to Cramer et al., U.S. Pat. No. 7,112,621 to Rohrbaugh et al.; co-pending U.S. patent application Ser. Nos. 10/338,603 and 10/338,610 by Cramer et al.; Ser. No. 13/005,237 by Lu et al.; and Ser. No. 13/428,404 by Xu et al.

Some polymers used for nonwoven fiber production may be inherently hydrophobic, and for certain applications they may be surface treated or coated with various agents to render them hydrophilic. A surface coating may include a surfactant coating. One such surfactant coating is available from Schill & Silacher GmbH, Böblingen, Germany, under the Tradename Silastol PHP 90.

Another way to produce nonwovens with durably hydrophilic coatings, is via applying a hydrophilic monomer and a radical polymerization initiator onto the nonwoven, and conducting a polymerization activated via UV light resulting in monomer chemically bound to the surface of the nonwoven as described in co-pending U.S. Patent Publication No. 2005/0159720.

Another way to produce hydrophilic nonwovens made predominantly from hydrophobic polymers such as polyolefins is to add hydrophilic additives into the melt prior to extrusion.

Another way to produce nonwovens with durably hydrophilic coatings is to coat the nonwoven with hydrophilic nanoparticles as described in co-pending applications U.S. Pat. No. 7,112,621 to Rohrbaugh et al. and in PCT Application Publication WO 02/064877.

Typically, nanoparticles have a largest dimension of below 750 nm. Nanoparticles with sizes ranging from 2 to 750 nm may be economically produced. An advantage of nanoparticles is that many of them can be easily dispersed in water solution to enable coating application onto the nonwoven, they typically form transparent coatings, and the coatings applied from water solutions are typically sufficiently durable to exposure to water. Nanoparticles can be organic or inorganic, synthetic or natural. Inorganic nanoparticles generally exist as oxides, silicates, and/or carbonates. Typical examples of suitable nanoparticles are layered clay minerals (e.g., LAPONITE™ from Southern Clay Products, Inc. (USA), and Boehmite alumina (e.g., Disperal P2™ from North American Sasol. Inc.). According to one example, a suitable nanoparticle coated non-woven is that disclosed in the co-pending patent application Ser. No. 10/758,066 entitled “Disposable absorbent article comprising a durable hydrophilic core wrap” by Ponomarenko and Schmidt.

In some cases, the nonwoven web surface can be pre-treated with high energy treatment (corona, plasma) prior to application of nanoparticle coatings. High energy pre-treatment typically temporarily increases the surface energy of a low surface energy surface (such as PP) and thus enables better wetting of a nonwoven by the nanoparticle dispersion in water.

Notably, hydrophilic non-wovens are also useful in other parts of an absorbent article. For example, topsheets and absorbent core layers comprising permanently hydrophilic non-wovens as described above have been found to work well.

A nonwoven also may include other types of surface coating. In one example, the surface coating may include a fiber surface modifying agent that reduces surface friction and enhances tactile lubricity. Preferred fiber surface modifying agents are described in U.S. Pat. Nos. 6,632,385 and 6,803,103; and U.S. Pat. App. Pub. No. 2006/0057921.

According to one example, the nonwoven may comprise a material that provides good recovery when external pressure is applied and removed. Further, according to one example, the nonwoven may comprise a blend of different fibers selected, for example from the types of polymeric fibers described above. In some embodiments, at least a portion of the fibers may exhibit a spiral curl which has a helical shape. According to one example, the fibers may include bicomponent fibers, which are individual fibers each comprising different materials, usually a first and a second polymeric material. It is believed that the use of side-by-side bi-component fibers is beneficial for imparting a spiral curl to the fibers.

In order to enhance softness perceptions of the absorbent article, nonwovens forming the backsheet may be hydroenhanced or hydroengorged. Hydroenhanced/hydroengorged nonwovens are described in U.S. Pat. Nos. 6,632,385 and 6,803,103, and U.S. Pat. App. Pub. No. 2006/0057921.

A nonwoven may also be treated by a “selfing” mechanism. By “selfing” nonwovens, high densities of loops (>150 in 2) may be formed which protrude from the surface of the nonwoven substrate. Since these loops act as small flexible brushes, they create an additional layer of springy loft, which may enhance softness. Nonwovens treated by a selfing mechanism are described in U.S. Pat. App. Pub. No. US 2004/0131820.

Any of the nonwoven types described herein may be used for the topsheet, backsheet, outer layer, barrier cuff, loops component in a hook-and-loop fastening system of an absorbent article, or any other portion of a manufactured article such as cleansing wipes and other personal hygiene products, dusters and dusting cloths, household cleaning cloths and wipes, laundry bags, dryer bags and sheets comprising a layer formed of nonwoven web.

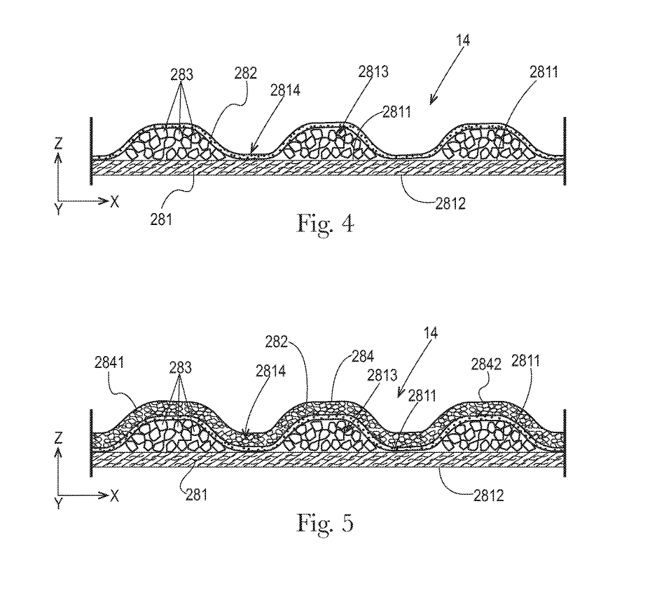

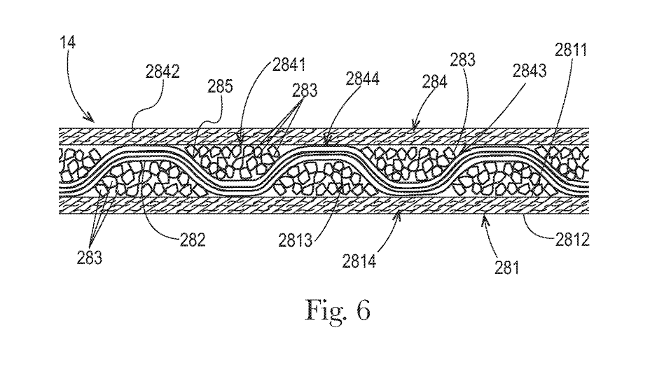

In one embodiment, an absorbent article includes an absorbent core 14 that is substantially cellulose free, as described in U.S. Pat. No. 7,750,203; U.S. Pat. No. 7,744,576, and U.S. Patent Publication No. 2008/0312617A1. Cross-sectional views of examples of suitable absorbent cores are schematically represented in FIGS. 4-6. In one embodiment, an absorbent core 14 comprises first and second layers of material 281, 282 and an absorbent material 283 disposed between the first and second layers 281, 282. In one embodiment the first and second layers of material can be a fibrous material chosen from at least one of a nonwoven fibrous web, a woven fibrous web and a layer of thermoplastic adhesive material. Although the first and second layers can be made of a same material, in one embodiment, the first layer 281 is a nonwoven fibrous web and the second layer 282 is a layer of thermoplastic adhesive material. A nonwoven fibrous web 281 can include synthetic fibers, such as mono-constituent fibers of PE, PET and PP, multi-constituent fibers such as side by side, core/sheath or island in the sea type fibers. Such synthetic fibers may be formed via a spunbonding process or a meltblowing process. The nonwoven fibrous web 281 may include a single layer of fibers but it may also be advantageous to provide the nonwoven web with multiple layers of fibers such as multiple layers of spunbond fibers, multiple layers of meltblown fibers or combinations of individual layer(s) of spunbond and meltblown fibers. In one embodiment, the nonwoven web 281 can be treated with an agent (such as a surfactant) to increase the surface energy of the fibers of the web. Such an agent renders the nonwoven web more permeable to liquids such as urine. In another embodiment, the nonwoven web can be treated with an agent (such as a silicone) that lowers the surface energy of the fibers of the nonwoven web. Such an agent renders the nonwoven web less permeable to liquids such as urine.

The first layer 281 comprises a first surface 2811 and a second surface 2812 and at least regions 2813 of the first surface are in direct facial relationship with a significant amount of absorbent material 283. In one embodiment an absorbent material is deposited on the first surface 2811 in a pattern to form regions 2813 on the first layer 281, which are in direct facial relationship with a significant amount of absorbent polymer material 283 and regions 2814 on the first web that are in facial relationship with only an insignificant amount of absorbent material. By “direct facial relationship with a significant amount of absorbent material” it is meant that some absorbent material is deposited on top of the regions 2813 at a basis weight of at least 100 g/m², at least 250 g/m² or even at least 500 g/m². The pattern may include regions that all have the same shape and dimensions (i.e. projected surface area and/or height). In the alternative the pattern may include regions that have different shape or dimensions to form a gradient of regions.

In one embodiment, the second layer 282 is a layer of a thermoplastic adhesive material. “Thermoplastic adhesive material” as used herein is understood to mean a polymer composition from which fibers are formed and applied to the absorbent material with the intent to immobilize the absorbent material in both the dry and wet state. Non-limiting examples of thermoplastic adhesive material may comprise a single thermoplastic polymer or a blend of thermoplastic polymers. The thermoplastic adhesive material may also be a hot melt adhesive comprising at least one thermoplastic polymer in combination with other thermoplastic diluents such as tackifying resins, plasticizers and additives such as antioxidants. In certain embodiments, the thermoplastic polymer has typically a molecular weight (Mw) of more than 10,000 and a glass transition temperature (Tg) usually below room temperature or −6° C.>Tg<16° C. In certain embodiments, typical concentrations of the polymer in a hot melt are in the range of about 20 to about 40% by weight. Exemplary polymers are (styrenic) block copolymers including A-B-A triblock structures, A-B diblock structures and (A-B)n radial block copolymer structures wherein the A blocks are non-elastomeric polymer blocks, typically comprising polystyrene, and the B blocks are unsaturated conjugated diene or (partly) hydrogenated versions of such. The B block is typically isoprene, butadiene, ethylene/butylene (hydrogenated butadiene), ethylene/propylene (hydrogenated isoprene), and mixtures thereof. Other suitable thermoplastic polymers that may be employed are metallocene polyolefins, which are polymers prepared using single-site or metallocene catalysts. In exemplary embodiments, the tackifying resin has typically a Mw below 5,000 and a Tg usually above room temperature, typical concentrations of the resin in a hot melt are in the range of about 30 to about 60% by weight, and the plasticizer has a low Mw of typically less than 1,000 and a Tg below room temperature, with a typical concentration of about 0 to about 15%.

The thermoplastic adhesive material 282 can be disposed substantially uniformly within the absorbent material 283. In the alternative, the thermoplastic adhesive material 282 can be provided as a fibrous layer disposed on top of the absorbent material 283 and the regions 2814 of the first surface 2811 that are in facial relationship with only an insignificant amount of absorbent material. In one embodiment, a thermoplastic adhesive material is applied at an amount of between 1 and 20 g/m², between 1 and 15 g/m² or even between 2 and 8 g/m². The discontinuous deposition of absorbent material on the first layer 281 imparts an essentially three-dimensional structure to the fibrous layer of thermoplastic material 282. In other words, the layer of thermoplastic adhesive material follows the topography resulting from the absorbent material 283 deposited on the first nonwoven fibrous web 281 and the regions 2814 that only include insignificant amounts of absorbent material. Without intending to be bound by any theory, it is believed that the thermoplastic adhesive materials disclosed herein enhance immobilization of the absorbent material in a dry and wet state.

In one embodiment, the absorbent core 14 may further comprise a second layer of a nonwoven fibrous material 284. This second layer may be provided of the same material as the nonwoven fibrous layer 281, or in the alternative may be provided from a different material. It may be advantageous for the first and second nonwoven fibrous layers 281, 284 to be different in order to provide these layers with different functionalities.

The regions 2813 may have any suitable shape in the x-y dimension of the absorbent core. In one embodiment, the regions 2813 form a pattern of disc that are spread on the first surface of the first web 281. In one embodiment, the regions 2813 form a pattern of longitudinal “strips” that extend continuously along the longitudinal axis of the absorbent core (i.e. along the y dimension). In an alternative embodiment, these strips may be are arranged to form an angle of at between 10 and 90 degrees, between 20 and 80 degrees, between 30 and 60 degrees, or even 45 degrees relative to the longitudinal axis of the absorbent article.

In one embodiment, the second nonwoven layer 284 has a first surface 2841 and a second surface 2842 and an absorbent material 283 applied to its first surface 2841 in order to form a pattern of regions 2843 that are in direct facial relationship with a significant amount of absorbent material 283 and regions 2844 on the first surface 2841 that are in facial relationship with only an insignificant amount of absorbent material as previously discussed. In one embodiment, a thermoplastic adhesive material 285 may further be applied on top of the second nonwoven layer 284 as previously discussed in the context of the first web/absorbent material/thermoplastic adhesive material composite. The second nonwoven layer 284 may then be applied on top of the first nonwoven layer 281. In one embodiment, the pattern of absorbent material present on the second nonwoven layer 284 may be the same as the pattern of absorbent material present on the first nonwoven layer 281. In another embodiment, the patterns of absorbent material that are present on the first and second nonwoven layers are different in terms of at least one of the shape of the regions, the projected surface areas of the regions, the amount of absorbent material present on the regions and the type of absorbent material present on the regions.

The absorbent core 14 may also comprise an auxiliary adhesive which is not illustrated in the figures. The auxiliary adhesive may be deposited on at least one of or even both the first and second nonwoven layers 281, 284 before application of the absorbent material 283 in order to enhance adhesion of the absorbent material as well as adhesion of the thermoplastic adhesive material 282, 285 to the respective nonwoven layers 281, 284. The auxiliary adhesive may also aid in immobilizing the absorbent material and may comprise the same thermoplastic adhesive material as described hereinabove or may also comprise other adhesives including but not limited to sprayable hot melt adhesives, such as H.B. Fuller Co. (St. Paul, Minn.) Product No. HL-1620-B. The auxiliary adhesive may be applied to the nonwoven layers 281, 284 by any suitable means, but according to certain embodiments, may be applied in about 0.5 to about 1 mm wide slots spaced about 0.5 to about 2 mm apart. Non-limiting examples of suitable absorbent material 283 include absorbent polymer material such as cross linked polymeric materials that can absorb at least 5 times their weight of an aqueous 0.9% saline solution as measured using the Centrifuge Retention Capacity test (Edana 441.2-01). In one embodiment, the absorbent material 283 is absorbent polymer material which is in particulate form so as to be flowable in the dry state.

The present invention is directed to absorbent articles comprising multiple nonwoven materials having matching visible elements. Specifically, matching the physical and/or graphic patterns of the nonwoven materials of the topsheet/inner liner, and/or backsheet/outer cover, and/or leg cuff material can enhance the overall air flow and breathability understanding of the absorbent article and generate enhanced consumer visual appeal. Matching visible elements and patterns better connote overall flow of air and humidity, as consumers understand that all elements of the absorbent article work together in a holistic, harmonious fashion.

Matching the physical and/or graphic patterns of the nonwoven materials is impactful to enhance the understanding of breathability and air flow between absorbent article components, as caregivers recognize that for something to flow there needs to be a pathway or connection to follow. In absorbent articles that do not have matching physical and/or graphic patterns on the nonwoven materials, there is no recognition of air flow or breathability between the nonwoven layers; even if there is a physical or graphic pattern on one nonwoven material, there is no potential connection to the other nonwoven materials in the absorbent article. When the physical and/or graphic nonwoven patterns match, there is a recognition that air flow and coordinated breathability exist between the nonwoven materials.

In one embodiment of the present invention, two of more nonwoven materials present in the absorbent article have matching visible elements (100) (as defined herein) that are repeated on the surface of the nonwoven materials. Matching visible elements may be exactly the same, or they may be rotated, mirrored, reduced in size, enlarged in size, and/or altered in aspect ratio between the nonwoven materials and still be considered a matching visible element. Thus, in one embodiment, the matching visible element is the same size on the nonwoven materials. In one embodiment, the matching visible element is reduced in sized or enlarged in size between the nonwoven materials.

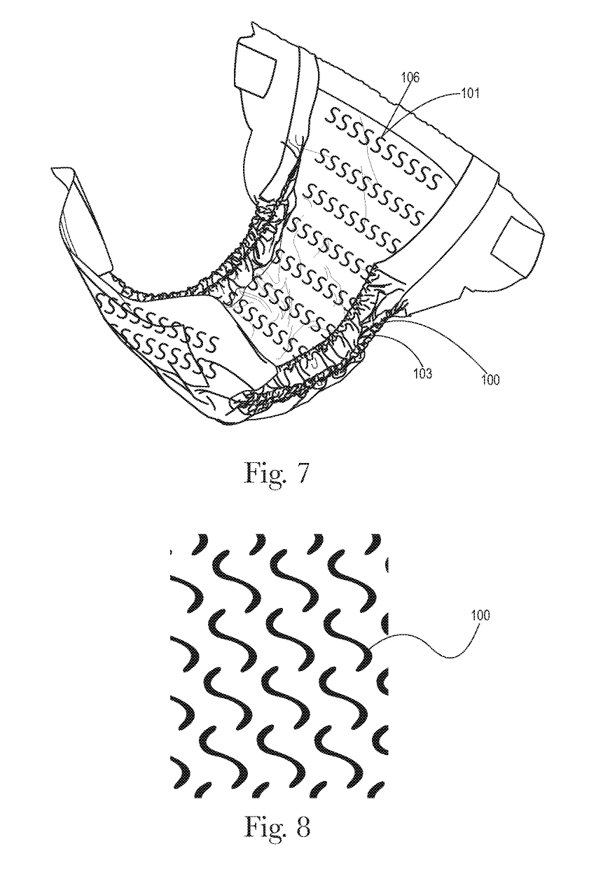

Further, the matching visible element (100) may be partially altered such that at least about 50%, at least about 60%, at least about 70%, at least about 80%, at least about 90% of the visible element is the same on both nonwoven materials and still be considered a matching visible element. In one embodiment, the matching visible elements (100) are 100% the same on both nonwoven materials. One example of an absorbent article having visible elements present on multiple nonwovens is shown in FIG. 7. In FIG. 7, the interior topsheet, exterior backsheet, and leg cuff material (pattern not shown) all have matching visible elements (100).

Further, the matching visible element (100) may include a repeat unit cell, wherein one cell repeats at least one time on the nonwoven material. In such an embodiment, the repeat unit cell may be rotated, mirrored, reduced in size, enlarged in size, and/or altered in aspect ratio between the nonwoven materials and still be considered a matching visible element.

In addition, the matching visible element (100) may have the same or different color on the nonwoven materials and still be considered a matching visible element. Further, the background to the visible element (100) may have the same of different color as the visible element (100). Those skilled in the art may select matching colors from a color wheel.

The color wheel was created by arranging red, orange, yellow, green, cyan blue, indigo and violet in a circle of natural progression. Primary colors of pigment are blue, yellow, and red representing thirds on the color wheel. These are the building blocks of color. They cannot be broken down into other colors, nor can any other colors combine to create them. The secondary colors are created by complement the primary colors. In between each of the primary colors lies the shade created by the combination of the two, for example blue and yellow create green, yellow and red create orange, and red and blue create violet. The tertiary colors are created by mixing one secondary color and one primary color (yellow green, for example) and lie in between their respective primary and secondary colors, and directly across from their complements.

Matching colors on the visible element or as the background of the visible element can be either selected from opposite ends of the color wheel to bring out each color and appear pleasing to the eyes. Alternatively, it can be selected from neighboring colors on the color wheel to create a calming effect and play off each other in a harmonious fashion.

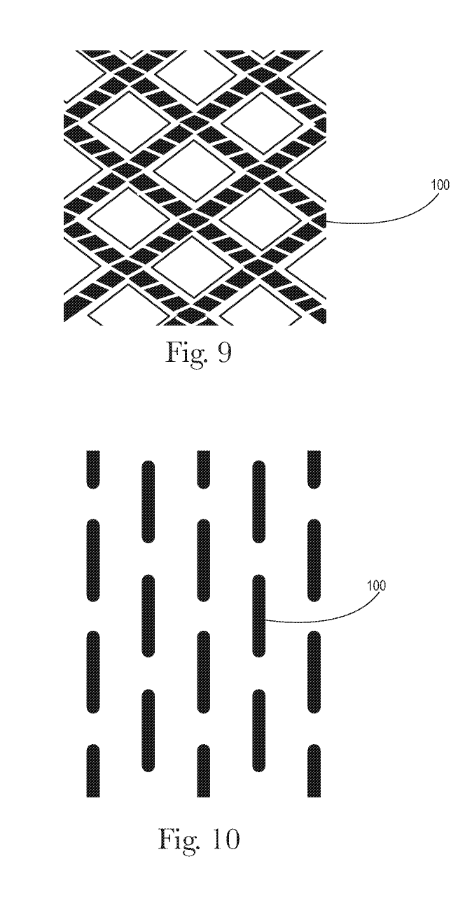

Non-limiting examples of visible elements (100) that may be used in the present invention are shown in FIGS. 8-10.

In one embodiment, the topsheet/inner cover comprises a first nonwoven material having a first visible element (101) and the backsheet/outer cover comprises a second nonwoven material having a second visible element (102), such that the first visible element (101) and the second visible element (102) are matching visible elements. In one embodiment, the backsheet/outer cover comprises a second nonwoven material having a second visible element (102) and the leg cuff comprises a third nonwoven material having a third visible element (103), such that the second visible element (102) and the third visible element (103) are matching visible elements. In one embodiment, the topsheet/inner cover comprises a first nonwoven material having a first visible element (101) and the leg cuff comprises a third nonwoven material having a third visible element (103), such that the first visible element (101) and the third visible element (103) are matching visible elements. In one embodiment, the topsheet/inner cover comprises a first nonwoven material having a first visible element (101), the backsheet/outer cover comprises a second nonwoven material having a second visible element (102), and the leg cuff comprises a third nonwoven material having a third visible element (103), such that the first visible element (101), the second visible element (102) and the third visible element (103) are all matching visible elements. In one embodiment, absorbent article components, that may or may not be nonwoven materials have matching visible elements. Such components include but are not limited to side panels, waist feature, fastening system, fastening members, landing zone, films, laminates, etc.

In one embodiment, the visible element is formed by any suitable embossing technique known in the art. In one embodiment, the visible element is formed by any suitable printing technique known in the art. In one embodiment, the visible element is formed by any suitable thermobonding technique known in the art. In one embodiment, the visible element is formed by any suitable calendar-bonding technique known in the art. In one embodiment, the visible element is formed by at least two techniques, including, but not limited to embossing, printing, thermobonding, calendar-bonding, and combinations thereof. In one embodiment, the visible element is formed by one technique on one component of the absorbent article and different technique on another component of the absorbent article.

The dimensions and values disclosed herein are not to be understood as being strictly limited to the exact numeral values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising a topsheet comprising a first nonwoven material, a backsheet comprising a second nonwoven material, and an absorbent core disposed between the topsheet and backsheet, and matching visible elements, wherein the matching visible elements comprise a first visible element disposed on the first nonwoven material and a second visible element disposed on the second nonwoven material; and wherein the matching visible elements are formed by a method selected from the group consisting of thermobonding, calendar-bonding, and combinations thereof.

2. The absorbent article of claim 1 wherein the matching visible elements further comprise a third visible element.

3. The absorbent article of claim 2 wherein the third visible element is disposed on a third nonwoven material.

4. The absorbent article of claim 1 wherein the matching visible elements are exactly the same.

5. The absorbent article of claim 1 wherein the matching visible elements are rotated, mirrored, reduced in size, enlarged in size, and/or altered in aspect ratio.

6. The absorbent article of claim 5, wherein the first visible element is rotated with respect to the second visible element.

7. The absorbent article of claim 5, wherein a visible portion of the first visible element is rotated with respect to the comparable portion of the second visible element.

8. The absorbent article of claim 5, wherein at least one of the first visible element or the second visible element is reduced in size or enlarged in size as compared to the other.

9. The absorbent article of claim 5, wherein at least a visible portion of the first visible element is reduced in size or enlarged in size as compared to a portion of the second visible element.

10. The absorbent article of claim 5, wherein at least one of the first visible element or the second visible element is altered in aspect ratio as compared to the other.

11. The absorbent article of claim 5, wherein at least a visible portion of the first visible element is altered in aspect ratio as compared to a portion of the second visible element.

12. The absorbent article of claim 1 wherein at least one of the first visible element or the second visible element is partially altered such that at least about 50% of the first visible element and the second visible element are the same.

13. The absorbent article of claim 1, wherein the first visible element and the second visible element include a repeat unit cell.

14. The absorbent article of claim 1, wherein the first visible element and the second visible element are the same in color.

15. The absorbent article of claim 1, wherein the first visible element and the second visible element are different in color.

16. An absorbent article comprising a topsheet, a backsheet and an absorbent core disposed between said topsheet and said backsheet; the absorbent article further comprising two or more article components having matching visible elements, wherein the matching visible elements comprise a first visible element disposed on the first article component and a second visible element disposed on the second article component; and wherein the matching visible elements are formed by embossing.

17. The absorbent article of claim 16 wherein the matching visible elements are exactly the same.

18. The absorbent article of claim 16 wherein the matching visible elements are rotated, mirrored, reduced in size, enlarged in size, and/or altered in aspect ratio.

19. The absorbent article of claim 16 wherein the two or more article components are selected from the group consisting of: side panels, a waist feature, a fastening system, fastening members, a landing zone, films, laminates, the topsheet, the backsheet, and leg cuffs.

20. The absorbent article of claim 16 wherein the two or more article components each comprise a nonwoven material.
